(19) United States
(12) Patent Application Publication
(10) Pub. No.: US 2016/0060615 A1
(43) Pub. Date: Mar. 3, 2016

(54) DEVICE FOR THE IDENTIFICATION, SEPARATION AND / OR CELL TYPE-SPECIFIC MANIPULATION OF AT LEAST ONE CELL OF A CELLULAR SYSTEM

(71) Applicants: Thomas Walther, Cork (IE); Michiel Postema, Bergschenhoek (NL)

(72) Inventors: Thomas Walther, Cork (IE); Michiel Postema, Bergschenhoek (NL)

(21) Appl. No.: 14/476,187

(22) Filed: Sep. 3, 2014

(51) Int. Cl. C12N 13/00; C12M 1/42; C12M 1/00; C12M 1/34; C12N 5/073; C12N 5/071

(52) U.S. Cl. CPC C12N 13/00; C12N 5/0603; C12N 5/0686; C12M 47/04; C12M 47/02; C12M 41/46; C12M 35/04; C12N 2521/10

(57) ABSTRACT

The invention, in part, relates to devices for the identification, separation, and/or cell type-specific manipulation of at least one cell of a cellular system.

### TECHNICAL FIELD

The invention relates to devices for the identification, separation and/or cell type-specific manipulation of at least one cell of a cellular system.

### BACKGROUND OF THE INVENTION

Cell cultures play a major role in many areas of the life sciences, especially in biotechnology and biomedical research. Cell cultures are used in the diagnosis and treatment of a variety of diseases, both in humans and in animals. Due to the growing reservations about animal testing, the establishment of biologically relevant in vitro test systems, and thus cell cultures, is becoming more important. Primary cells are the basis for the development of complex in vitro models and test methods (assays) and therefore offer an excellent alternative to animal testing. The preparation of primary cultures from a tissue sample as well as their further cultivation for experimental purposes represents a major challenge.

For this purpose, the cell type-specific separation of the cells is of crucial importance in order to obtain clean primary cell cultures. In the current state of the art, the cells must be separated by flow cytometry. A disadvantage of this technique is that the specific cells must be selectively stained, for example with fluorescent dyes, which is not always possible so that the cells cannot be distinguished from each other. A further possibility consists in the selection of cells by specific and labelled antibodies. However, for many cell types, no specific antibodies are available. This method is also very time consuming and costly. Furthermore, this method cannot be employed for cells which are not free-floating in a cell culture. This is especially true for cells in a multi-cell system, as a tissue, organ, or multicell system. Different cell types can be sorted by morphological studies. However, this method depends very much on the skill and the experience of the person who performs this separation. Furthermore, this method is extremely time-consuming. The cells themselves must be removed from the multi-cell system, so that the sterile cell culture conditions are compromised. In organisms, this kind of manipulations is often not feasible without fatally damaging the organism.

It is known that particles or particles in a liquid, e.g. encapsulated microbubbles, have a different resonance frequency in sonication waves than free gas bubbles in the liquid, and can be therefore discriminated (Postema M. Fundamentals of Medical Ultrasonics. Spon Press, London, 2011). Based on their acoustic properties, microbubbles are also suitable as ultrasound contrast agent for applications such as diagnostic imaging. Here, particles with same acoustic properties attract each other, while particles with different acoustic properties repel each other. The mutual attraction of particles with same acoustic properties can lead to the fusion of such micro gas bubbles. This phenomenon can be explained by the secondary Bjerknes forces.

The publication Kotopoulis S., Postema M. Microfoam formation in a capillary. Ultrasonics 2010; 50:206-268 describes the formation of a microfoam through the manipulation of microbubbles with ultrasound. Due to the excitation of microbubbles with ultrasound, the individual micro gas bubbles begin to attract and in this way form a cluster consisting of micro gas bubbles. This happens immediately, already in the first second of the action of ultrasound. If the thus formed microbubbles clusters continue to be exposed to the ultrasound, than the resulting clusters begin to form larger clusters arranged together and thus form a micro-foam. As long as the micro-foam for annealed clusters is in the ultrasonic wave field, the clusters behave as a unit.

Furthermore, the publication Jönsson H., C. Holm, A. Nilsson, F. Petersson, P. Johnsson, Laurell T. Ultrasound can radically reduce embolic load to brain after cardiac surgery. Ann. Thorac. Surg. 2004; 78:1572-1578 demonstrates the separation of particles in liquids using acoustic standing waves. In a specific example it is shown how lipid particles may be separated and removed by means of ultrasound from blood and other present compartments (plasma, blood cell, sugar, etc.). This allows the prevention of microemboli caused by increased lipid content in the blood and especially those that occur after cardiac surgery (bypass surgery).

Each cell type and any microorganism should have a specific acoustic behavior, which depends on its compressibility, density, and geometry. Each cell type or microorganism thus responds most strongly to a characteristic sound frequency. This characteristic sound frequency is referred to as its specific resonance frequency. If a cell is exposed to a sound wave of its specific resonant frequency, the cell responds with a dynamic signal in the form of an oscillation. However, if a certain amplitude of this resonant frequency is exceeded, the cell or microorganism can vibrate so strongly that it is destroyed. The publication Delalande A., Kotopoulis S., Rovers T., Pichon C., Postema M. Sonoporation at low mechanical index. Bubble Science, Engineering and Technology 2011; 3:3-11 shows the acoustic activity of certain cancer cells.

WO 01/00084 A1 discloses an apparatus and method for non-invasive destruction of (tumor) tissue by treatment of the tissue with acoustic shock waves generated by predetermined frequencies. These shock waves cause only the seriously degenerated tissue to vibrate, thus protecting the surrounding tissue. The amplitude of the corresponding shock wave is so high that the mechanical friction in the tissue is high enough for the destruction of cells by cavitation and heating. The down side is that all the irradiated cells are destroyed. Furthermore, the shock waves penetrate deep into the tissue which can also be damaged.

U.S. Pat. No. 6,406,429 discloses an apparatus and a method for non-invasive detection of cystic structures and precursors of cancer cells in all tissues by ultrasonic waves. It is an imaging technique. Methods for the elimination of individual cell types are not disclosed.

The publication Kotopoulis S., A. Schommartz, Postema M. Sonic cracking of blue-green algae. Applied Acoustics 2009; 70:1306-1312 describes how ultrasonic waves of frequency, as used in the clinical diagnostic range (200 kHz to 2.2 MHz) can be used to destroy the heterocysts in blue-green algae (cyanobacteria). The irradiated algae, which now lack their buoyancy units, begin to sediment. The sedimented algae continue to have perfectly intact chloroplasts, which remain unaffected by the action of ultrasound. It is therefore possible to manipulate or destroy specific cells with ultrasound, while other cell types in the environment are not affected.

The object of the invention is therefore to provide an apparatus and a method for the identification, separation and/or cell type-specific manipulation (Sonopulation) of at least one cell of a cellular system as well as of microorganisms. Said manipulation can also comprise the selective killing of cells, at least one cell of a cellular system, as well as of microorganisms without compromising the sterile cell culture conditions or having to remove the cells from the cell system.

### SUMMARY OF THE INVENTION

According to one aspect of the invention, a device (1) for the identification, separation and/or cell type-specific manipulation of at least one cell of a cellular system as well as of microorganisms, is provided. The device (1) includes an ultrasound transmitter (5), a control unit (2) and a receiving unit (3), wherein the ultrasound transmitter (5) includes a piezo-electric component, which is controlled with a frequency greater than 5 MHz, and has a narrow bandwidth. In some embodiments, the piezoelectric member is a piezoelectric crystal, a piezoelectric ceramic or a piezoelectric polymer. In some embodiments, the piezoelectric component has a resonance of 7 MHz per millimeter. In some embodiments, the piezoelectric component is a wafer which is cut in an orientation of 36° to the Y-axis of a lithium niobate (LiNbO₃) crystalline lattice. In some embodiments, the control unit (2) has a magnification lens (6), and comprises a binocular or an inverted microscope. In some embodiments, the receiving unit (3) is a cell culture dish, petri dish (4) or the like.

According to another aspect of the invention, methods for one or more of the identification, separation and cell type-specific manipulation of at least one cell of a cellular system as well as of microorganisms with any embodiment of an aforementioned device (1) are provided. The methods include steps of a) introducing the cell system into the receiving unit (3) of the device (1), and sonicating the cell system with the Ultrasound transmitter (5) with a cell type-specific frequency. In some embodiments, the method further comprises at least one of the following steps: c) distinguishing and sorting of cells of the multi-cell system or the cell system in primary cell cultures; d) creating a resonance profile for the sonicated cells of the multi-cell system or the cell system; e) transforming the sonicated cells of the multi-cell system or the cell system; f) transfecting the sonicated cells of the multi-cell system or the cell system; g) discriminating the cells of the multi-cell system or the cellular system based on their genotype, wherein the cells are sperm; h) isolating and destroying cells of the multi-cell system, or the cellular system, wherein the cells are viral or parasite bearing cells or tumor cells; i) acting on the cells of the multi-cell system, or the cellular system to prevent restenosis, where the cells are cells of the neointima; j) isolating and identifying different subpopulations within the multi-cell system or the cell system, the multi-cell system or the cell system comprising microorganisms in mixed populations; k) activating specific dendritic cells; and l) manipulating intracellular signalling.

According to yet another aspect of the invention, use of any embodiment of an aforementioned device (1) is provided for performing a method for the identification, separation and/or cell type-specific manipulation of at least one cell of a cellular system as well as of microorganisms, the method comprising the steps of: a) introducing the cell system into the receiving unit (3) of the device (1), and b) sonicating the cell system with the Ultrasound transmitter (5) with a cell type-specific frequency. In some embodiments, the method also includes at least one of the following steps: c) distinguishing and sorting of cells of the multi-cell system or the cell system in primary cell cultures; d) creating a resonance profile for the sonicated cells of the multi-cell system or the cell system; e) transforming the sonicated cells of the multi-cell system or the cell system; f) transfecting the sonicated cells of the multi-cell system or the cell system; g) discriminating the cells of the multi-cell system or the cellular system based on their genotype, wherein the cells are sperm; h) isolating and destroying cells of the multi-cell system, or the cellular system, wherein the cells are viral or parasite bearing cells or tumor cells; i) acting on the cells of the multi-cell system, or the cellular system to prevent restenosis, where the cells are cells of the neointima; j) isolating or identifying different sub-populations within the multi-cell system, or the cellular system, wherein the multi-cell system, or the cell system comprises microorganisms in mixed populations; k) activating specific dendritic cells; and l) manipulating intracellular signalling.

The invention provides a device for the identification, separation and/or cell type-specific manipulation of at least one cell of a cellular system as well as of microorganisms comprising a Ultrasound transmitter, a control unit and a receiving unit, wherein the Ultrasound transmitter comprises a piezo-electric component, which emits a controllable narrow-bandwidth frequency greater than 5 MHz.

With the device, a fast and convenient separation and identification of different cell types in a multi-cell system is made possible without damaging the individual cells or the sterile cell culture conditions. Removal of the individual cells of the multi-cell system is not necessarily required. This is very important especially for primary cell cultures. And manipulating (Sonopulation), such as transfection or transformation of individual cell types in a cell system or a multi-cell system can be performed in this way. With the control unit one can observe directly, when necessary, the success of this manipulation. Furthermore, this device can determine the resonant frequency of a particular cell type and precisely target this specific resonance frequency for cell type-specific manipulation. Particularly in cells that are infected with viruses or other parasites, this presents an opportunity to separate these cells from the cell culture and subsequently remove them or, if appropriate, to destroy these cells without affecting or damaging other cells.

In certain embodiments of the device according to the invention, the piezoelectric member is a piezoelectric crystal, a piezoelectric ceramic, or a piezoelectric polymer. The oldest and best known piezoelectric components for ultrasonic generation are piezoelectric crystals. This includes for example the stable a-quartz modification, lithium niobate or gallium orthophosphate. More piezo-electric crystals are berlinite, tourmalines and Seignette's salt. Piezoelectric ceramics include, for example, lead titanate (PT), lead zirconate titanate (PZT), bismuth titanate, barium titanate and lead metaniobate (PMN). The most widely used piezoelectric polymers include polyvinylidene fluoride (PVDF) or a copolymer of polyvinylidene fluoride and trifluoroethylene. Thus, the piezoelectric component can be optimally adapted to the required design or use and thus optimally adapted to the respective requirements (coupling factor, cross-coupling, acoustic impedance (acoustic impedance and bandwidth).

Furthermore, it is advantageous if the piezo-electric device has a resonance of 7 MHz per millimeter, so as to obtain an optimum conversion of electrical signals to mechanical signals. Piezo-electric components with a specific resonance frequency show, when excited by a voltage at this resonant frequency, the highest amplitude of the generated ultrasound. Thus, the resonance frequency should be in the frequency range in which the ultrasonic frequency is needed. A large scatter of the resonance frequencies would mean a deterioration of the issued ultrasonic signal (sound pressure). However, other resonances of the piezo-electric device are also conceivable.

In a further advantageous embodiment, the piezoelectric component is a wafer which is cut into an orientation of 36° to the Y-axis of a lithium niobate (LiNbO₃) crystalline lattice. Lithium niobate is a piezoelectric crystal, which is not broken even at a high applied voltage, and thus is suitable for the generation of ultrasound in high frequency ranges (250 kHz to 40 MHz). Furthermore, lithium niobate has a very high resonance. In certain embodiments of the invention, the piezo-electric wafer is made of lithium niobate (LiNbO₃) and has a thickness of 0.5 mm and a diameter of 7.6 cm. Such wafers are provided by e.g. Boston Piezo-Optics. Inc., Boston, Mass., USA.

To study or observe the separation and/or cell type-specific manipulation of at least one cell of a cell system and micro-organisms, it is a great advantage if the control unit of the device according to the invention is a microscope with magnifying optics, which in certain embodiments of the invention may be a binocular inverted microscope. Thus, during irradiation of the sample with ultrasonics, the results can be checked immediately and the ultrasonic irradiation reduced to a minimum in order to exclude possible damage to other cells.

It is advantageous if the receiving unit is a support for a cell culture dish, petri dish, or the like. Especially then, the to be identified, separated or manipulated cells can be brought into the device, without taking them from their surrounding medium, whereby the sterile environment is maintained. Also a possibly harmful contact with air and oxygen can be excluded if the cells can remain in the nutrient medium. In particular for cell cultures, this can be beneficial as cell cultures are usually created in petri dishes. However, other sample types can also be easily and conveniently introduced into cell culture dishes or Petri dishes. Also the introduction of petri dishes into cell culture cabinets or incubators is possible.

In a method for the identification, separation and/or cell type-specific manipulation of at least one cell of a cellular system as well as of microorganisms with a device according to the invention, the invention provides that the method comprises the steps of

a) Introduction of the cell system into the receiving unit of the apparatus and

b) Sonication of the cell system with the Ultrasound transmitter with a cell type-specific frequency.

It is particularly advantageous when the method comprises at least one of the following steps

Distinguishing and sorting of cells of the multi-cell system or the cell system in primary cell cultures;

Creation of a resonance profile for the sonicated cells of the multi-cell system or the cell system;

Transformation of the sonicated cells of the multi-cell system or the cell system;

Transfection of the sonicated cells of the multi-cell system or the cell system;

Discrimination of the cells of the multi-cell system or the cellular system based on their genotype, wherein the cells are preferably sperm;

Isolation and destruction of cells of the multi-cell system, or the cellular system, wherein the cells are preferably viral or parasite bearing cells or tumor cells;

Acting on the cells of the multi-cell system, or the cellular system to prevent restenosis, where the cells are preferably cells of the neointima;

Isolation or identification of different sub-populations within the multi-cell system, or the cellular system, wherein the multi-cell system, or the cell system preferably comprises microorganisms in mixed populations;

Activation of specific dendritic cells;

Manipulation of intracellular signalling.

The inventive method is therefore suitable to separate cells of a multi-cell system according to different cell types. This is particularly important in cell cultures, and particularly in primary cell cultures. The cells in the cell culture can be sorted and separated quickly and accurately. Furthermore, a response profile can be created for the cells of the multi-cell system whereby only these specific cell types may be influenced or manipulated by the ultrasound frequency.

Furthermore, the targeted transformation or transfection of a particular cell type in a multi-cell system using ultrasound is possible with the method without having to make a manual separation of the cell types. Cells, which in some embodiments of the invention may be sperm can be discriminated against based on the genotype. Another advantage of the method is that special cell types, in particular virus or parasite-infected cells or otherwise degenerated cells, particularly tumor cells, with exposure to ultrasonic waves of a specific resonance frequency of these cells in the multi-cell system can be isolated and destroyed. The remaining cells are unaffected in the multi-cell system.

In order to avoid restenosis after a treated vasoconstriction the treatment can be performed with ultrasound. This is especially important for the cells of the neointima. The isolation or identification of subpopulations of different cell types within a multi-cell system can be performed with the inventive method. Thus, the method offers a lot of advantages and new opportunities for the identification, separation, and/or cell type-specific manipulation of different cells in a multi-cell system. Activation of dendritic cells which are cells of the immune system and one of the key cell types in inflammatory processes, is used to generate an immune response or to mobilize the immune system. The manipulation of intracellular signalling processes also allows for the stimulation of the proliferation of stem or progenitor cells with specific diseases that require regenerative processes for wound healing. By influencing the intracellular signalling processes within the cell is also possible to initiate apoptosis and initiate the targeted cell death. This is particularly important in cancer research.

With the optimal adjustment of the acoustic parameters relating to one type of cell and the subsequent irradiation of the cells with these sound frequencies, the permeability of the cell membrane can be increased, whereby an improved uptake of drugs, contrast agents, etc. is facilitated without undesirable side-effects in, for example, the intracellular signalling processes or cell physiological processes.

The invention further provides the use of the inventive device for carrying out the method according to the invention.

The use of the method comprises at least one of the steps

Distinguishing and sorting of cells of the multi-cell system or the cell system in primary cell cultures;

Creation of a resonance profile for the sonicated cells of the multi-cell system or the cell system;

Transformation of the sonicated cells of the multi-cell system or the cell system;

Transfection of cells of the multi-cell system or the cell system treated with ultrasound;

Discrimination of the cells of the multi-cell system or the cellular system based on their genotype, wherein in some embodiments of the invention the cells are sperm;

Isolation and destruction of cells of the multi-cell system, or the cellular system, wherein in some embodiments of the invention, the cells are viral or parasite bearing cells or tumor cells;

Acting on the cells of the multi-cell system, or the cellular system to prevent restenosis, wherein in some embodiments of the invention, the cells are cells of the neointima;

Isolation or identification of different sub-populations within the multi cell system or cell system, whereby in some embodiments of the invention, the multi-cell system, or the cell system comprises microorganisms in mixed populations;

Activation of specific dendritic cells;

Manipulation of intracellular signalling.

### BRIEF DESCRIPTION OF THE DRAWINGS

FIG. 1 shows a diagram of an embodiment of a structure of a device for the identification, separation and/or cell type-specific manipulation of at least one cell of a cellular system, as well as microorganisms;

FIG. 2 provides photomicrographic images and cells. FIG. 2A shows an image of CHO cells before and after treatment with ultrasound. FIG. 2B shows an image of HEK cells before and after treatment with ultrasound.

Further features, details and advantages of the invention will become apparent from the wording of the claims and from the following description of embodiments with reference to the drawings.

### DETAILED DESCRIPTION OF THE INVENTION

### Definitions

Microorganism: micro-organisms, microscopic unicellular or multicellular organisms, such as bacteria, protozoa, fungi, yeasts and algae.
Cell system:

a) mono cell system: Each organic system containing only one type of cell; or

b) multi-cell system: Each organic system containing more than one type of cell, such as cell cultures or organisms.

Primary cell culture: Not immortalized (i.e., mortal) cell culture, which was obtained directly from a tissue.
Ultrasound transmitter: A component which generates acoustic signals in the ultrasonic range.
Sonopulation: alteration or manipulation of cells by sonic (acoustic waves).
Cell type: Cells which perform the same function in an organism. Therefore, they are also similar mostly in their appearance, their structure and their internal structure, and intracellular signalling. However, individual protozoa can be seen as a special type of cell.
The terms “cell” and “cellular” may be used interchangeably herein.

### LIST OF REFERENCE NUMERALS

1 Device
2 Control unit
3 Receiving unit
4 Petri dish
5 Ultrasound transmitter
6 Magnification optics

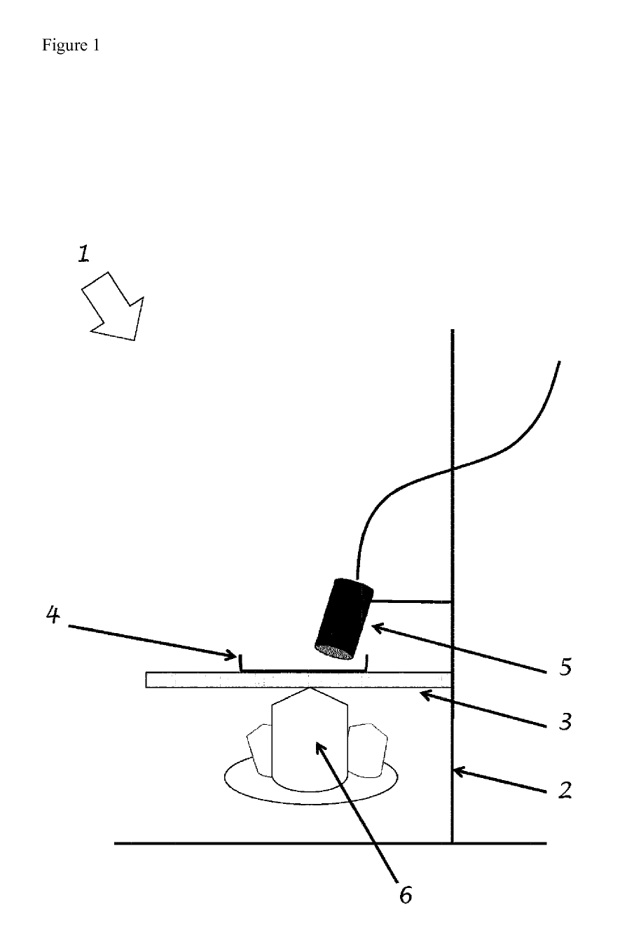

The generally designated device according to the invention (FIG. 1) for the identification, separation and/or cell type-specific manipulation of at least one cell of a cell system as well as of microorganisms has a control unit 2, and a biological inverted microscope 6 (MBL 3200, A. Kruss Optronic GmbH, Hamburg, Germany). On the control unit 2, a receiving unit 3 is installed for a Petri dish 4. The Petri dish 4 has a diameter of 10 cm and is filled with cells detached from the bottom of the dish 4 in Dulbecco's Modified Eagle Medium (DMEM)—a standardized culture medium for cell cultures. The cells in the petri dish 4 are, for example, Chinese hamster ovary (Cricetulus griseus) (CHO) cells, human embryonic kidney cells (HEK cells), endothelial cells of the aorta of cattle (BAEC cells), or embryonic Mouse fibroblast cells (3T3 his/NIH cells).

The ultrasound is generated by an Ultrasound transmitter 5, wherein the Ultrasound transmitter 5 comprises a piezo-electric component, which is controlled with a frequency greater than 5 MHz, and has a narrow bandwidth. The Ultrasound transmitter 5 consists of a 7 MHz transducer having a piezo-electric crystal as a piezoelectric component, which may also be a piezoelectric ceramic or a piezoelectric polymer. The piezoelectric crystal is made of lithium niobate (LiNbO₃), which is cut with an orientation of 36° to the Y axis and having a resonance of 7 MHz per millimeter. This Ultrasound transmitter 5 is arranged at an angle of 17° above the petri dish 4.

A not shown AFG3102 frequency generator (Tektronix, Everett, Wash., USA) controls the Ultrasound transmitter 5 with a fundamental frequency of a continuous wave. The signal of the not shown frequency generator is routed through a 20 dB attenuator (not shown) before it is directed as an input signal to a also not shown power amplifier 2100L 50 dB RF (Electronics & Innovation Ltd., Rochester, N.Y., USA), and there finally comes to the Ultrasound transmitter 5, where the ultrasound generated is conducted into the sample in the petri dish 4.

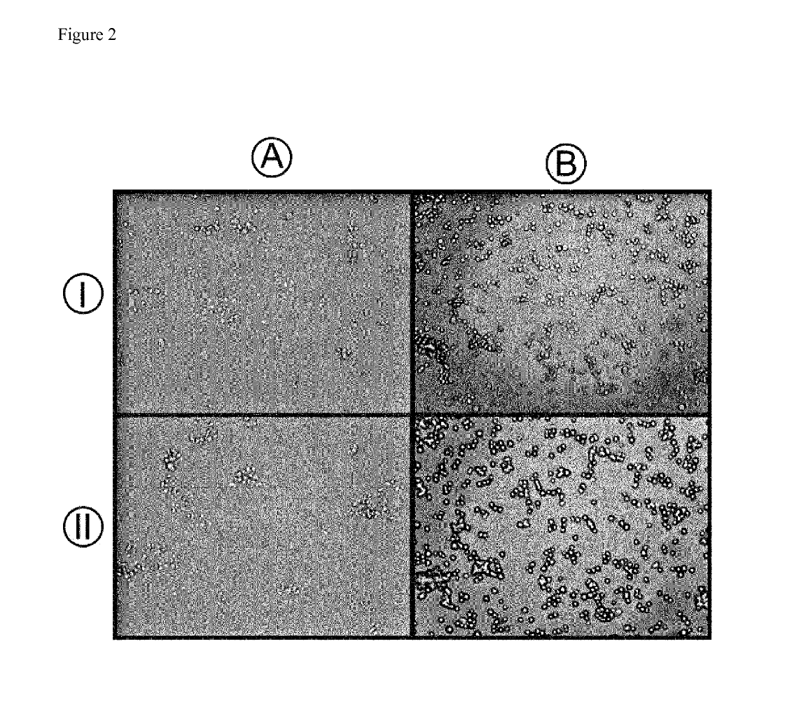

FIG. 2A shows Chinese hamster ovary (CHO) cells in a petri dish 4. The top image (2 AI) shows the cells from the bottom of the dish placed in a nutrient medium prior to sonication. The lower picture (2 A II) shows the same sample after a treatment of 30 seconds with ultrasound at a frequency of 7 MHz. The cells have assembled into larger and more densely packed clusters. This clustering has already started after a few seconds of sonication.

FIG. 2B shows human embryonic kidney cells (HEK cells) in a petri dish 4 which have previously been detached from the bottom of the dish 4 and are freely movable in the nutrient medium. The upper frame (2 Bi) showing the cells in the broth before the ultrasound treatment. The image (B 2 II) shows the cells after 30 seconds ultrasound treatment at a frequency of 7 MHz. These cells, as opposed to CHO cells, show no cluster formation. Different cell types therefore show different behavior upon ultrasonic treatment with the same frequency. Each of these images represents a sample area of 960×720 (microns).

A mixture of both cell types (CHO cells and HEK cells) in a sample dish 4 also shows a clustering, with only the same types of cells forming a cluster (not shown).

The invention is not limited to the embodiments described above, but can be modified in many ways.

All of the claims, the description and the drawing features and advantages, including construction details, spatial arrangements and process steps can be inventive per se and in various combinations.

The contents of all literature references, patents, and published patent applications cited throughout this application are incorporated herein by reference in their entirety.

We claim:

1. A device (1) for the identification, separation and/or cell type-specific manipulation of at least one cell of a cellular system as well as of microorganisms, wherein the device (1) has a Ultrasound transmitter (5), a control unit (2) and a receiving unit (3), wherein the Ultrasound transmitter (5) comprises a piezo-electric component, which is controlled with a frequency greater than 5 MHz, and has a narrow bandwidth.

2. The device of claim 1, wherein the piezoelectric member is a piezoelectric crystal, a piezoelectric ceramic or a piezoelectric polymer.

3. The device of claim 1, wherein the piezoelectric component has a resonance of 7 MHz per millimeter.

4. The device (1) of claim 1, wherein the piezoelectric component is a wafer which is cut in an orientation of 36° to the Y-axis of a lithium niobate (LiNbO₃) crystalline lattice.

5. The device (1) of claim 1, wherein the control unit (2) has a magnification lens (6), and comprises a binocular or an inverted microscope.

6. The device (1) of claim 1, wherein the receiving unit (3) is a cell culture dish, petri dish (4) or the like.

7. A method for the identification, separation and/or cell type-specific manipulation of at least one cell of a cellular system as well as of microorganisms with a device (1) of claim 1, comprising the steps of
a) introducing the cell system into the receiving unit (3) of the device (1), and
b) sonicating the cell system with the Ultrasound transmitter (5) with a cell type-specific frequency.

8. The method of claim 7, wherein the method further comprises at least one of the following steps:
c) distinguishing and sorting of cells of the multi-cell system or the cell system in primary cell cultures;
d) creating a resonance profile for the sonicated cells of the multi-cell system or the cell system;
e) transforming the sonicated cells of the multi-cell system or the cell system;
f) transfecting the sonicated cells of the multi-cell system or the cell system;
g) discriminating the cells of the multi-cell system or the cellular system based on their genotype, wherein the cells are sperm;
h) isolating and destroying cells of the multi-cell system, or the cellular system, wherein the cells are viral or parasite bearing cells or tumor cells;
i) acting on the cells of the multi-cell system, or the cellular system to prevent restenosis, where the cells are cells of the neointima;
j) isolating and identifying different subpopulations within the multi-cell system or the cell system, the multi-cell system or the cell system comprising microorganisms in mixed populations;
k) activating specific dendritic cells; and
l) manipulating intracellular signalling.

9. Use of a device (1) of claim 1 for performing a method for the identification, separation and/or cell type-specific manipulation of at least one cell of a cellular system as well as of microorganisms, the method comprising the steps of:
a) introducing the cell system into the receiving unit (3) of the device (1), and
b) sonicating the cell system with the Ultrasound transmitter (5) with a cell type-specific frequency.

10. The use according to claim 9, wherein the method further comprises at least one of the following steps:
c) distinguishing and sorting of cells of the multi-cell system or the cell system in primary cell cultures;
d) creating a resonance profile for the sonicated cells of the multi-cell system or the cell system;
e) transforming the sonicated cells of the multi-cell system or the cell system;
f) transfecting the sonicated cells of the multi-cell system or the cell system;
g) discriminating the cells of the multi-cell system or the cellular system based on their genotype, wherein the cells are sperm;
h) isolating and destroying cells of the multi-cell system, or the cellular system, wherein the cells are viral or parasite bearing cells or tumor cells;
i) acting on the cells of the multi-cell system, or the cellular system to prevent restenosis, where the cells are cells of the neointima;
j) isolating or identifying different sub-populations within the multi-cell system, or the cellular system, wherein the multi-cell system, or the cell system comprises microorganisms in mixed populations;
k) activating specific dendritic cells; and
l) manipulating intracellular signalling.
